(19) United States
(12) Patent Application Publication
(10) Pub. No.: US 2004/0082058 A1
(43) Pub. Date: Apr. 29, 2004

(54) Array hybridization apparatus and method for making uniform sample volumes

(76) Inventors: Arthur Schleifer, Portola Valley, CA (US); Allen C. Thompson, San Francisco, CA (US); Duane M. Dutton, San Jose, CA (US); Joseph A. Worrall III, Lomond, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/283,450

(22) Filed: Oct. 29, 2002

(51) Int. Cl. C12M 1/34

(52) U.S. Cl. 435/287.2; 435/293.1; 435/288.3

(57) ABSTRACT

An apparatus and method for making array hybridization chambers with uniform sample volumes. The apparatus employs a spacer to define a hybridization chamber of uniform volume between a slide, substrate, and gasket after the slide, substrate, spacer and gasket have been contacted. The spacer is non deformable or partially deformable and will only allow the adjacent gasket to a similar height or volume. Methods of making the hybridization apparatus and chambers are also disclosed.

### FIELD OF THE INVENTION

The invention relates to the field of micro arrays, and more particularly to assay chambers for improved hybridization binding. In particular, the invention relates to an array hybridization apparatus having a chamber with uniform sample volume defined by a spacer. Methods of making the apparatus and chambers are also described.

### BACKGROUND OF THE INVENTION

Polynucleotide arrays (such as DNA or RNA arrays) are known and are used, for example, as diagnostic or screening tools. Such arrays include regions of usually different sequence polynucleotides arranged in a predetermined configuration on a substrate. These regions (sometimes referenced as “features”) are positioned at respective locations (“addresses”) on the substrate. In use, the arrays, when exposed to a sample, will exhibit an observed binding or hybridization pattern. This binding pattern can be detected upon interrogating the array. For example, all polynucleotide targets (for example, DNA) in the sample can be labeled with a suitable label (such as a fluorescent dye), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated by depositing previously obtained biopolymers (such as from synthesis or natural sources) onto a substrate, or by in situ synthesis methods. Methods of depositing obtained biopolymers include dispensing droplets to a substrate from dispensers such as pin or capillaries (such as described in U.S. Pat. No. 5,807,522) or such as pulse jets (such as a piezoelectric inkjet head, as described in PCT publications WO 95/25116 and WO 98/41531, and elsewhere). For in situ fabrication methods, multiple different reagent droplets are deposited from drop dispensers at a given target location in order to form the final feature (hence a probe of the feature is synthesized on the array stubstrate). The in situ fabrication methods include those described in U.S. Pat. No. 5,449,754 for synthesizing peptide arrays, and described in WO 98/41531 and the references cited therein for polynucleotides. The in situ method for fabricating a polynucleotide array typically follows, at each of the multiple different addresses at which features are to be formed, the same conventional iterative sequence used in forming polynucleotides from nucleoside reagents on a support by means of known chemistry. This iterative sequence is as follows: (a) coupling a selected nucleoside through a phosphite linkage to a functionalized support in the first iteration, or a nucleoside bound to the substrate (i.e. the nucleoside-modified substrate) in subsequent iterations; (b) optionally, but preferably, blocking unreacted hydroxyl groups on the substrate bound nucleoside; (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in a known manner.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere

Polynucleotide arrays have previously been provided in two formats. In one format, the array is provided as part of a package in which the array itself is disposed on a first side of a glass or other transparent substrate. This substrate is fixed (such as by adhesive) to a housing with the array facing the interior of a chamber formed between the substrate and housing. An inlet and outlet may be provided to introduce and remove sample and wash liquids to and from the chamber during use of the array. The entire package may then be inserted into a laser scanner, and the sample exposed array may be read through a second side of the substrate.

In another format, the array is present on an unmounted glass or other transparent slide substrate. This array is then exposed to a sample optionally using a temporary housing to form a chamber with the array substrate. The slide may then be placed in a laser scanner to read the exposed array.

Each of these arrays hybridization chambers are problematic. The first type of array hybridization chamber has a uniform sample chamber, but is not easy to assemble and often requires the application of adhesives or glues to attach the substrate or slides to a fixed array housing. Pre-assembled hybridization chamber are more difficult to load in that they require an inlet and outlet septum and needle to puncture the septum to allow the sample to enter the chamber and another needle to allow gas to exit the chamber as it is filled. In contrast, the hybridization chambers that employ a resilient gasket are often easier to assemble, but suffer from the limitation that the array hybridization chamber is not uniform in volume or height. The variability of height in the hybridization chamber can affect the overall performance of array hybridizations or binding experiments.

It, therefore, would be desirable to provide an array hybridization apparatus with uniform sample volume or height that is cost effective and easy to assemble or make.

### SUMMARY OF THE INVENTION

The invention provides an array hybridization apparatus and method of making the same. The array hybridization apparatus, comprises a slide for holding an array; a substrate opposite the slide for acting as a backing for the array hybridization apparatus; a gasket interposed between the slide and the substrate; and a spacer interposed between the slide and the substrate and adjacent to the gasket. An array hybridization chamber of uniform volume is defined between the first substrate, the substrate, the gasket and the spacer when the slide and the substrate contact the gasket and the spacer. The uniform array hybridization chamber is a result of the gasket being more deformable than the spacer positioned adjacent to it. The spacer will only allow the slide and substrate to compress the gasket to the spacer's height.

The invention also provides a method for making an array hybridization apparatus having an array hybridization chamber of uniform volume comprising, providing an array slide, a substrate, a gasket and a spacer; and contacting the slide, substrate, gasket and spacer to define a uniform array hybridization chamber there between.

### BRIEF DESCRIPTION OF THE DRAWINGS

Embodiments of the invention will now be described with reference to the drawings, in which:

FIG. 1 illustrates a slide carrying an array, of the present invention;

FIG. 2 is an enlarged view of a portion of FIG. 1 showing ideal spots or features;

FIG. 3 is an enlarged illustration of a portion of the substrate in FIG. 2;

FIG. 4 is an exploded perspective view of the present invention showing how the array hybridization apparatus is assembled;

FIG. 5A shows a first embodiment of the invention;

FIG. 5B shows a second embodiment of the invention;

FIG. 6 shows a cross sectional view of the present invention when it is in assembled form.

FIG. 7A shows an enlarged portion of FIG. 6 showing the gasket and spacer.

FIG. 7B shows the same view as FIG. 7A, but the gasket and spacer have been contacted by the slide and substrate.

### DETAILED DESCRIPTION OF THE INVENTION

Before describing the invention in detail, it must be noted that, as used in this specification and the appended claims, the singular forms “a,” “an,” and “the” include plural referents unless the context clearly dictates otherwise. Thus, for example, reference to “a gasket” includes more than one “gasket”. Reference to a “spacer” or “substrate” includes more than one “spacer” or “substrate”. In describing and claiming the present invention, the following terminology will be used in accordance with the definitions set out below.

A “biopolymer” is a polymer of one or more types of repeating units. Biopolymers are typically found in biological systems (although they may be made synthetically) and particularly include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A “peptide” is used to refer to an amino acid multimer of any length (for example, more than 10, 10 to 100, or more amino acid units). A biomonomer fluid or biopolymer fluid reference a liquid containing either a biomonomer or biopolymer, respectively (typically in solution).

A “set” or “sub-set” of any item (for example, a set of features) may contain one or more than one of the item (for example, a set of clamp members may contain one or more such members). An “array”, unless a contrary intention appears, includes any one, two or three dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one that is to be evaluated by the other (thus, either one could be an unknown mixture of polynucleotides to be evaluated by binding with the other). An “array layout” refers collectively to one or more characteristics of the features, such as feature positioning, one or more feature dimensions, and some indication of a moiety at a given location. “Hybridizing” and “binding”, with respect to polynucleotides, are used interchangeably. When one item is indicated as being “remote” from another, this is referenced that the two items are at least in different buildings, and may be at least one mile, ten miles, or at least one hundred miles apart.

The term “adjacent” or “adjacent to” refers to a component or element that is near, next to or adjoining. For instance, a gasket may be adjacent to a spacer.

The term “substantially deformable”, “compressible” or “deformable” shall all have a similar meaning.

The term “slide” refers to any number of materials having at least one planar surface capable of contacting a gasket or spacer. The term shall be broad based to include substrates, polymeric materials, silica based materials, plastics etc.. It's important that the “slide” maintain a certain amount of rigidity to compress or deform the gasket and contact the spacer. In certain instances a “slide” will be transparent to allow light to pass through its medium. However, this is not required. Also, the “slide” must be capable in certain instances to allow for the mounting or construction of an array on its surface. Although in certain cases this will not be required if the array is constructed on a separate surface.

The term “substrate” shall refers to any number of materials well know in the art that are capable of acting as a mounting medium.

It will also be appreciated that throughout the present application, that words such as “front”, “rear”, “back”, “leading”, “trailing”, “top”, “upper”, and “lower”, are all used in a relative sense only. “Fluid” is used herein to reference a liquid. Reference to a singular item, includes the possibility that there are plural of the same items present. Furthermore, when one thing is “slid” or “moved” or the like, with respect to another, this implies relative motion only such that either thing or both might actually be moved in relation to the other.

All patents and other cited references are incorporated into this application by reference.

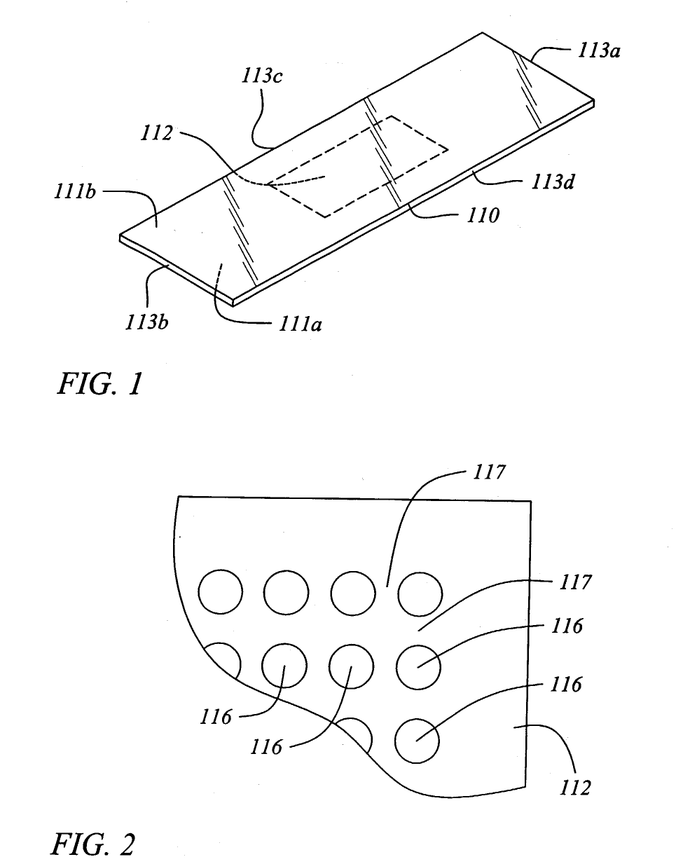

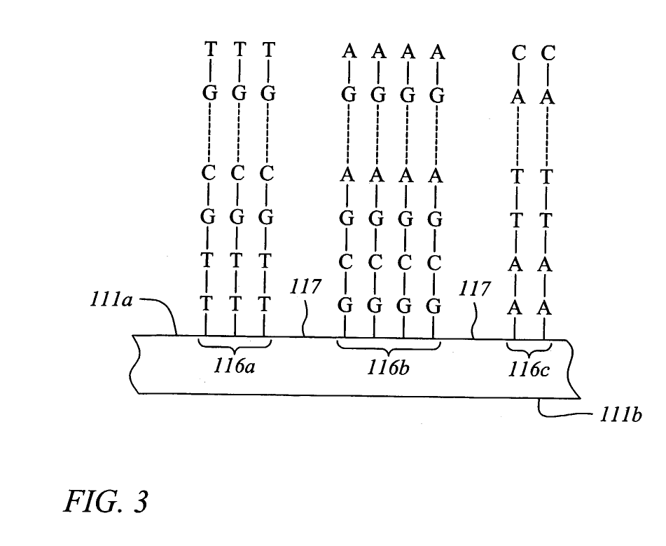

Referring first to FIGS. 1-3, typically the methods and apparatus of the present invention generate or use a contiguous planar transparent slide 110 carrying an array 112 disposed on a rear surface 111a of a substrate 110. It will be appreciated though, that more than one array (any of which are the same or different) may be present on the rear surface 111a, with or without spacing between such arrays. Note that one or more of the arrays 112 together will cover the entire regions of the rear surface 111a, with regions of the rear surface 111a adjacent to the opposed sides 113c, 113d and the leading end 113a and the trailing end 113b of the slide 110. A front surface 111b of the slide 110 does not carry any of the arrays 112. Each of the arrays 112 can be designed for testing against any type of sample, whether a trial sample, reference sample, a combination of them, or a known mixture of polynucleotides (in which latter case the arrays may be composed of features carrying unknown sequences to be evaluated). The slide 110 may be of any shape, and any holder used with it adapted accordingly, although the slide 110 will typically be rectangular in practice. The array 112 contains multiple spots or features 116 of biopolymers in the form of polynucleotides. A typical array may contain from more than ten, more than one hundred, more than one thousand or ten thousand features, or even more than from one hundred thousand features. All of the features 116 may be different, or some or all could be the same. In the case where the array 112 is formed by the conventional in situ or deposition of previously obtained moieties, as described above, by depositing for each feature at least one droplet of reagent such as by using a pulse jet such as an inkjet type head, interfeature areas 117 will typically be present which do not carry any polynucleotide. It will be appreciated though, that the interfeature areas 117 could be of various sizes and configurations. Each feature carries a predetermined polynucleotide (which includes the possibility of mixtures of polynucleotides). As per usual, A, C, G, T represent the usual nucleotides. It will be understood that there may be a linker molecule (not shown) of any known types between the rear surface 111a and the first nucleotide.

The slide 110 also carries on the front surface 111b, an identification code in the form of a bar code 115 printed on an opaque substrate in the form of a paper label attached by adhesive to the front side 111a (not shown in FIGS.). By “opaque” in this context is referenced that the means used to read the bar code 115 (typically a laser beam) cannot read the bar code 115 through the label without reading errors. Typically this means that less than 60% or even less than 50%, 30%, 20% or 10% of the signal from the code passes through the substrate. The bar code 115 contains an identification of the array 112 and either contains or is associated with, array layout or layout error information in a manner such as described in U.S. patent applications U.S. patent application Ser. Nos. 09/302,898 (filed Apr. 30, 1999) and 09/359,536 (filed Jul. 22, 1999; now issued as U.S. Pat. No. 6,180,351, Jan. 30, 2001) both originally assigned to Hewlett-Packard, incorporated herein by reference.

For the purposes of the discussions below, it will be assumed (unless the contrary is indicated) that the array 112 is a polynucleotide array formed by the deposition of previously obtained polynucleotides using pulse jet deposition units. However, it will be appreciated that an array of other polymers or chemical moieties generally, whether formed by multiple cycles in situ methods adding one or more monomers per cycle, or deposition of previously obtained moieties, or by other methods, may be present instead.

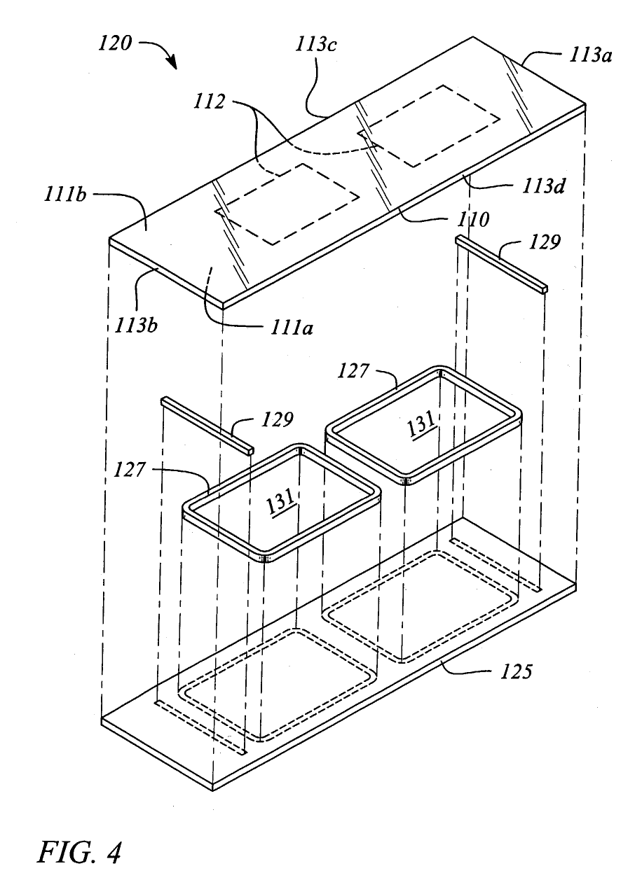

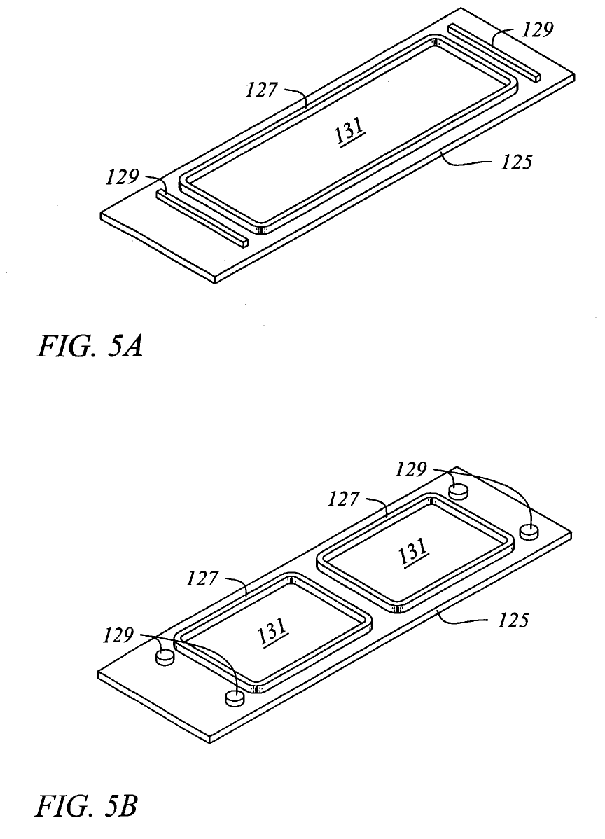

Referring now to FIGS. 4-5, the typical methods and apparatus of the present invention will now be described in more detail. An array hybridization apparatus 120 may comprise the slide 110 for holding the array 112, a substrate 125 opposite the slide 110 for acting as a backing for the array hybridization apparatus 120, a gasket 127 interposed between the slide 110 and the substrate 125, and a spacer 129. The spacer 129 is interposed between the slide 110 and the substrate 125 and positioned adjacent to the gasket 127 wherein the array hybridization chamber 131 of uniform volume is defined between the slide 110, the substrate 125, the gasket 127 and the spacer 129 when the slide 110 and the substrate 125 contact the gasket 127 and the spacer 129.

The hybridization apparatus 120 is designed for holding or positioning the array 112 so that maximum hybridizations/annealing of nucleic acids can take place between the array 112 and a target 113 of interest (target not shown in drawings).

The slide 110 may typically contain or be attached to the array 112 and may comprise any number of transparent materials such as glass, plastic, silicon or other materials known in the art to contain or capable of containing arrays. Slide 110 can be thought of as the array substrate, but need not contain the array 112. The array 112 could also be attached or part of the substrate 125. The slide 110 may be designed in a variety of shapes, sizes and widths.

The substrate 125 may typically comprise a material such as glass, plastic, silicon or other materials know in the art. The substrate 125 may be thought of as being the backing for the hybridization apparatus 120. However, in certain embodiments the substrate 125 may actually contain or comprise the array 112. The substrate 125 may be designed in a variety of shapes, sizes and widths.

The gasket 127 may be attached to the slide 110, the substrate 125 or both and is designed for holding or retaining the hybridization solutions for the array 112. Typically, the gasket 127 will be rectangular in shape and will be attached to the substrate 125. The shape and design of the gasket 127 is not important to the invention. However, it is important to the invention that the gasket maintains a sufficient compressibility so as to form a seal between the slide 110, the gasket 127 and the substrate 125 when they contact each other. The gasket 127 must also retain the hybridization solution when the slide 110, substrate 125, the gasket 127 and the spacer 129 are all contacted. The gasket 127 may comprise any number of materials that are substantially deformable. For instance, the gasket 125 may comprise materials such as rubber, silicon, silicone, acrylamides, polyacrylamides, non-synthetic polymers and synthetic polymers etc..

The spacer 129 may be attached to the slide 110, the substrate 125 or both. Typically, the spacer 129 will be attached to the slide 110 when the gasket 127 is attached to the substrate 125. The spacer 129 may comprise any number of shapes and sizes. It may also be positioned in any number of positions on the substrate or slide and may comprise substantially non deformable or non-compressable materials such as metal, wood, plastic etc.. It is important to the invention that the spacer 129 be somewhat rigid relative to the gasket 127. For instance, the spacer 129 needs to be less deformable or compressible relative to the gasket 127. This allows the gasket 127 to act as a seal but deform only to the extent of the height of the spacer 129. Since the spacer 129 does not further collapse or compress the height or volume of the array hybridization chamber 131 can be gauged. The spacer 129 can range in height of from 25 to 500 microns. This forms the uniform hybridization chamber 131 having a fixed volume based on the height of the spacer 129. The spacer 129 will similarly retain a height in the range of from 25 to 500 microns.

FIGS. 5A and 5B show two embodiment of the present invention. The gasket 127 and the spacer 129 have been mounted or attached to the substrate 125. The gasket 127 helps to form the array hybridization chamber 131. As mentioned one or more spacers 129 may be used and the spacer 129 may have a variety of shapes and sizes. FIG. SA and 5B show two types of spacers 129 (rectangular and round). Typically, as shown, the spacers 129 are positioned adjacent to the gasket 127. They may be positioned anywhere on the slide 110 or the substrate 125. FIGS. 5A and 5B show the spacer 129 positioned exterior to the gasket 127. These examples are for illustrative purposes only and the invention should not be interpreted to be limited to these embodiments only. The uniform array hybridization chamber 131 contains the array 112 and nucleic acids in a hybridization solution that is retained by the gasket 127. The uniform array hybridization chamber 131 provides for protection of the array and reproducibility of results.

Having described the apparatus of the invention, a description of the method of assembling or making the array hybridization apparatus is now in order.

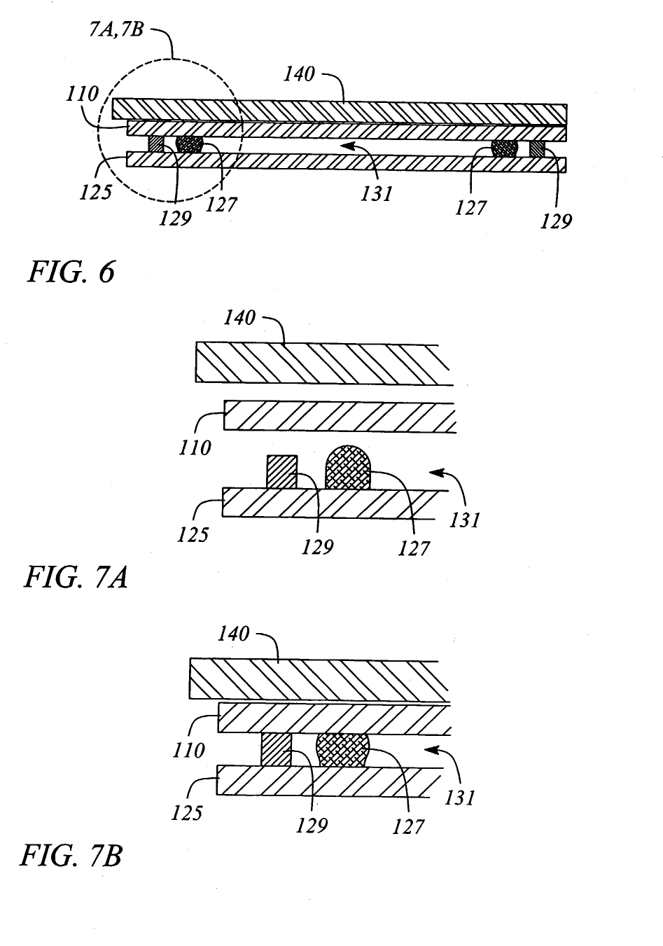

The array hybridization apparatus 120 can be easily assembled relative to other devices that contain fixed components. In its simplest form the array hybridization apparatus 120 may be constructed by providing the slide 110, the substrate 125, the gasket 127 and the spacer 129 and then contacting each of these components to define the uniform array hybridization chamber 131 (See FIG. 6). Typically, the gasket 127 and the spacer 129 are interposed between the slide 110 and the substrate 125. This may be accomplished by first attaching the gasket 127 to either or both of the substrates. Next the arrays or oligonucleotides are attached to the substrate 125 within the gasket 127 and the array solutions are then added. The spacer 129 may then be added, attached or constructed on the slide 110. The slide 110 with the attached spacer 129 and the substrate 125 with the attached gasket 127 may then be contacted to form the array hybridization chamber 131. The components may be contacted by clamping or by joining the slide 110, a cover 140 and the substrate 125 in any of a number of manners that are well known in the art. The components are contacted under enough pressure to form a sealed hybridization chamber 131 between the slide 110, the gasket 127 and the substrate 125. However, too much pressure can also destroy the array 112 and the array hybridization chamber 131 and this should be avoided. The spacer 129 helps prevent this problem.

FIG. 6 shows the present invention in its assembled form. The cover 140 is used to apply pressure to the front surface 111b of the slide 110. The substrate 125 will often be positioned on a rigid surface or placed in a container (not shown). As a result, the gasket 127 will be compressed to form a tight seal. As shown in the figures, the spacer 129 will not compress and the gasket 127 will only compress to the height of the spacer 129. In order for this to happen, the gasket 127 needs to be substantially more compressible or deformable than the spacer 129 and must also be slightly taller than the spacer 129 when mounted on the substrate 125 or slide 110 (See FIGS. 7A and 7B). The array hybridization chamber 131 that is formed in between, maintains a uniform volume or height and the array is not damaged.

Clearly, minor changes may be made in the form and construction of the invention without departing from the scope of the invention defined by the appended claims. It is not, however, desired to confine the invention to the exact form herein shown and described, but it is desired to include all such as properly come within the scope. claimed.

We claim:

1. An array hybridization apparatus, comprising:
(a) a slide for holding an array;
(b) a substrate opposite said slide for acting as a backing for said array hybridization apparatus;
(c) a gasket interposed between said slide and said substrate; and
(d) a spacer interposed between said slide and said substrate and adjacent to said gasket wherein an array hybridization chamber of uniform height is defined between said slide, said substrate, said gasket and said spacer when said slide and said substrate contact said gasket and said spacer.

2. An array hybridization apparatus as recited in claim 1, wherein said gasket comprises a deformable material.

3. An array hybridization apparatus as recited in claim 1, wherein said spacer comprises a substantially non-deformable material.

4. An array hybridization apparatus as recited in claim 1, wherein said gasket is attached to said slide.

5. An array hybridization apparatus as recited in claim 1, wherein said gasket is attached to said substrate.

6. An array hybridization apparatus as recited in claim 1, wherein said gasket is attached to both said slide and said substrate.

7. An array hybridization apparatus as recited in claim 1, wherein said spacer is attached to said slide.

8. An array hybridization apparatus as recited in claim 1, wherein said spacer is attached to said substrate.

9. An array hybridization apparatus as recited in claim 1, wherein said spacer is attached to both said slide and said substrate.

10. An array hybridization apparatus as recited in claim 1, wherein said spacer comprises a material selected from the group consisting of polyurethanes, plastics, acrylics, metals and non-deformable polymers.

11. An array hybridization apparatus as recited in claim 1, wherein said spacer is between 25 to 1000 microns in height.
An array hybridization apparatus as recited in claim 11, wherein said array hybridization chamber is between 25 to 1000 microns in height.

12. A method of making an array hybridization apparatus having an array hybridization chamber of uniform volume comprising:
(a) providing a slide, substrate, gasket and spacer; and
(b) contacting said slide, substrate, gasket and spacer to define a uniform array hybridization chamber.

13. A method of making an array hybridization apparatus with an array hybridization chamber of uniform volume, comprising:
(a) providing a slide opposite a substrate;
(b) interposing a gasket and spacer between said slide and said substrate;
(c) contacting said slide, said substrate, said gasket and said spacer to define a chamber there between.

14. A method of making an array hybridization apparatus with an array hybridization chamber of uniform volume, comprising:
(a) providing a slide;
(b) attaching a gasket to said slide;
(c) providing a substrate opposite said slide;
(d) applying a polymeric material to said substrate to define a spacer;
(e) contacting said slide, said gasket, said substrate, and said spacer to define said array hybridization chamber there between of uniform volume.
